(19) United States
(12) Patent Application Publication
(10) Pub. No.: US 2020/0178812 A1
(43) Pub. Date: Jun. 11, 2020

(54) PULSE PRESSURE CONDUCTING STRUCTURE, PORTABLE BLOOD PRESSURE DETECTION MODULE, AND SMART WEARABLE DEVICE

(71) Applicant: SHENZHEN KINGYIELD TECHNOLOGY CO., LTD., Shenzhen (CN)

(72) Inventors: Nianping Zhang, Shenzhen (CN); Dacheng Gong, Shenzhen (CN); Zhiming Huang, Shenzhen (CN)

(21) Appl. No.: 16/788,526

(22) Filed: Feb. 12, 2020

Related U.S. Application Data
(63) Continuation of application No. PCT/CN2018/071863, filed on Jan. 9, 2018.

(30) Foreign Application Priority Data
May 16, 2017 (CN) 201710343416.7

(51) Int. Cl. A61B 5/022; A61B 5/00; A61B 5/024; A61B 5/021

(52) U.S. Cl. CPC A61B 5/022; A61B 5/0024; A61B 5/02438; A61B 5/02141; A61B 2560/0431; A61B 5/742; A61B 5/6801; A61B 2562/02; A61B 5/7405

(57) ABSTRACT

A pulse pressure conducting structure comprises a housing and a pulse pressure transmission member, the pulse pressure transmission member joining with the housing to form a cavity for inflation, and at least a part of the pulse pressure transmitting member can, during inflation, directly or indirectly act on a body part to be measured of a subject to be detected. The pulse pressure transmitting member of such structure does not need to be wrapped around a limb, and the parts of the body at which it can be used are not limited. A portable blood pressure detection module and a smart wearable device are also disclosed.

### CROSS REFERENCE TO RELATED APPLICATIONS

The present application is a Continuation Application of PCT Application No. PCT/CN2018/071863 filed on Jan. 9, 2018, which claims the benefit of Chinese Patent Application No. 201710343416.7 filed on May 16, 2017. All the above are hereby incorporated by reference. The contents of which are incorporated herein by reference in their entirety.

### TECHNICAL FIELD

The present invention relates to a blood pressure detection device, particularly to a pulse pressure conducting structure for a blood pressure detection device.

### BACKGROUND OF THE INVENTION

Arterial blood pressure is the most important and commonly used measurement parameter in clinical practice, and blood pressure is also a component of risk scores for various important cardiovascular diseases, so it is medically important to measure blood pressure.

Currently, most non-invasive electronic sphygmomanometers use oscillography method, and a very few use auscultation method. Whether oscillography or auscultation method is used, the sphygmomanometers are cuff sphygmomanometers. The cuff includes a cloth bag and an air bag, and it is generally required that the length of the air bag is approximately 0.8 times that of the limbs, and the width of the air bag is 0.4 times that of the limbs. During blood pressure measurement, the cuff is wrapped about a limb to press against the arterial blood vessels of the limb. Blood pressure is detected by pressurizing the air bag in the cuff to obstruct arterial blood flow, and detecting changes in air pressure in the airbag or detecting Coriolis sound and air pressure in the air bag. Such cuff sphygmomanometers have a problem in that when in use, the cuff (air bag) needs to be wrapped about a limb, which is cumbersome and limits the parts of the body at which they can be used, Moreover, as a whole circumference of a wrist or arm is occupied, a device configured for simultaneous detection of vital sign parameters other than blood pressure, such as detection of heart rate by photoelectric method, is difficult to implement.

### SUMMARY OF THE INVENTION

The present application provides a novel pulse pressure transmission structure and a portable blood pressure detection module using the pulse pressure transmission structure, and further provides a smart wearable device using the pulse pressure transmission structure or the portable blood pressure detection module.

According to an aspect of the present application, an embodiment provides a pulse pressure transmitting structure for an electronic sphygmomanometer, which comprises a housing and a pulse pressure transmitting member, the pulse pressure transmitting member being mounted on the housing and joined with the housing or a fixing element fixed on the housing to form a cavity for inflation, and at least a part of the pulse pressure transmitting member protruding out of the housing, such that the pulse pressure transmitting member can, during inflation, directly or indirectly act on a body part to be measured of a subject to be detected.

As a further improvement of the pulse pressure transmitting structure, the pulse pressure transmitting member comprises an elastic portion made of an elastic material and/or a flexible portion made of a flexible material.

As a further improvement of the pulse pressure transmitting structure, at least one of the housing and the pulse pressure transmitting member has a recess, the housing and the pulse pressure transmitting member closing the recess to form the cavity.

As a further improvement of the pulse pressure transmission structure, an outer wall of the housing is recessed to form a first recess, and a side of the pulse pressure transmitting member facing the first recess is recessed to form a second recess. The pulse pressure transmitting member is pressed and sealed onto the first recess by a pressing member, such that the first recess and the second recess constitute the cavity for inflation.

As a further improvement of the pulse pressure transmitting structure, the pulse pressure transmitting member is mounted in the housing, and a part of the pulse pressure transmitting member protrudes from an inside of the housing to an outside of the housing.

As a further improvement of the pulse pressure transmitting structure, the part of the pulse pressure transmitting member protruding out of the housing is made of an elastic film or a flexible film.

According to an aspect of the present application, an embodiment provides a portable blood pressure detection module, comprising:

a housing having a mounting chamber;

a pulse pressure transmitting member mounted on the housing and joined with the housing to form a cavity for inflation, at least a part of the pulse pressure transmitting member protruding out of the housing, such that the pulse pressure transmitting member can, during inflation, directly or indirectly act on a body part to be measured of a subject to be detected;

an inflating and deflating structure in communication with the cavity and configured to inflate and deflate the cavity;

a detection unit configured to detect a pulse pressure signal in the cavity;

and a blood pressure calculation unit configured to calculate a blood pressure detection result based on the detected pulse pressure signal;

the inflating and deflating structure, the detection unit and the blood pressure calculation unit being mounted in the mounting chamber.

As a further improvement of the portable blood pressure detection module, a communication unit is further comprised. The communication unit is connected to the blood pressure calculation unit, and is configured to establish a communication connection between the portable blood pressure detection module and other devices.

As a further improvement of the portable blood pressure detection module, the communication unit includes a wireless transmission unit.

As a further improvement of the portable blood pressure detection module, the detection unit comprises an air pressure sensor, the air pressure sensor being in communication with the cavity and configured to detect a gas pressure signal in the cavity.

As a further improvement of the portable blood pressure detection module, the detection unit comprises a sound sensor and an air pressure sensor, the sound sensor being configured to detect Coriolis sound, and the air pressure sensor being configured to detect a cavity pressure signal.

As a further improvement of the portable blood pressure detection module, a display unit and/or a sound prompting unit are/is further comprised, the display unit and/or the sound prompting unit being connected to the blood pressure calculation unit and configured to display and/or prompt a detection result.

As a further improvement of the portable blood pressure detection module, the inflating and deflating structure comprises an air pump configured for pressurizing and a fast vent valve configured for venting, the air pump and the fast vent valve being in communication with the cavity.

As a further improvement of the portable blood pressure detection module, the inflating and deflating structure comprises an integrated air pump having an inflating and deflating function, the integrated air pump being in communication with the cavity.

As a further improvement of the portable blood pressure detection module, a mounting structure is further comprised, the mounting structure being fixedly connected to the housing and configured to mount the portable blood pressure detection module to other devices.

As a further improvement of the portable blood pressure detection module, the mounting structure includes at least one of an adhesion structure, a snap-in structure, a screw-in structure, and a magnetic adsorption structure.

As a further improvement of the portable blood pressure detection module, a wearing structure is further comprised, the wearing structure being fixedly connected to the housing and configured to wear the portable blood pressure detection module at an arterial blood vessel of a subject to be detected.

As a further improvement of the portable blood pressure detection module, the wearing structure includes at least one of a strap structure, a ring structure, a suction structure, and an adhesive structure.

As a further improvement of the portable blood pressure detection module, the wearing structure is formed by integrally extending from at least one side of the housing.

As a further improvement of the portable blood pressure detection module, the pulse pressure transmitting member includes an elastic portion made of an elastic material and/or a flexible portion made of a flexible material.

As a further improvement of the portable blood pressure detection module, at least one of the housing and the pulse pressure transmitting member has a recess, the housing and the pulse pressure transmitting member closing the recess to form the cavity.

As a further improvement of the portable blood pressure detection module, an outer wall of the housing is recessed to form a first recess, and a side of the pulse pressure transmitting member facing the first recess is recessed to form a second recess, and the pulse pressure transmitting member is pressed and sealed onto the first recess by a pressing member, such that the first recess and the second recess constitute a cavity for inflation.

As a further improvement of the portable blood pressure detection module, the pulse pressure transmitting member is mounted in the housing, and a part of the pulse pressure transmitting member protrudes from an inside of the housing to an outside of the housing.

As a further improvement of the portable blood pressure detection module, the part of the pulse pressure transmitting member protruding out of the housing is made of an elastic film or a flexible film

According to an aspect of the present application, an embodiment provides a smart wearable device, comprising:

a pulse pressure transmitting structure according to any one of the foregoing, configured to transmit pulse pressure of a subject to be detected;

an inflating and deflating structure configured to inflate and deflate a cavity;

a detection unit configured to detect a pulse pressure signal in the cavity;

a blood pressure calculation unit configured to calculate a blood pressure detection result according to the detected pulse pressure signal;

and a display unit and/or a sound prompting unit, the display unit and/or the sound prompting unit being connected to the blood pressure calculation unit and configured to display and/or prompt a detection result.

According to an aspect of the present application, an embodiment provides a smart wearable device, comprising a blood pressure detection module according to any one of the foregoing, configured to detect a pulse pressure of a subject to be detected.

The smart wearable device according to any one of the foregoing, further comprising:

a second communication unit configured to establish a wireless and/or wired communication connection with the blood pressure detection module;

and a display unit and/or a sound prompting unit configured to display and/or prompt a detection result.

As a further improvement of the smart wearable device, the smart wearable device includes one of a smart watch and a smart bracelet.

The portable blood pressure detection module according to the above embodiments uses a special pulse pressure transmission structure. The pulse pressure transmission structure comprises a housing and a pulse pressure transmission member, the pulse pressure transmission member joining with the housing or a fixing element fixed on the housing to form a cavity for inflation, and at least a part of the pulse pressure transmitting member can, during inflation, directly or indirectly act on a body part to be measured of a subject to be detected. The pulse pressure transmitting member of such structure does not need to be wrapped around a limb, and the parts of the body at which it can be used are not limited. What is only needed is to place the pulse pressure transmitting member on an arterial blood vessel. The pulse pressure will be transmitted by the pulse pressure transmitting member to be reflected in a gas change in the cavity, which allows a blood pressure detection film to detect a pulse pressure signal. The portable blood pressure detection module can not only be used at an artery on a wrist or an arm, but also be used at other body parts, such as a cervical artery and the heart. Moreover, since a large-volume airbag and a cuff are omitted, the portable blood pressure detection module can be made very small, which enables the portable blood pressure detection module to be not only individually attached to a body part to be measured, but also easily attached to other wearables, such as being attached to a strap of a watch. It also allows for a detection space for a detection device that detects other vital sign parameters.

The pulse pressure transmission structure or the portable blood pressure detection module can be miniaturized and can be integrated into a smart wearable device, such as a smart bracelet or a smart watch, so that blood pressure can be detected anytime and anywhere to track health status.

### BRIEF DESCRIPTION OF THE DRAWINGS

FIG. 1 is a schematic diagram showing a structure of a first embodiment of a portable blood pressure detection module of the present application;

FIG. 2 is a schematic diagram showing a usage of the first embodiment of a portable blood pressure detection module of the present application;

FIG. 3 is a cross-sectional view of the structure shown in FIG. 2;

FIGS. 4 and 5 are a schematic diagram showing a usage of the first embodiment of a portable blood pressure detection module of the present application;

FIG. 6 is a schematic diagram showing a structure of a second embodiment of a portable blood pressure detection module of the present application;

FIG. 7 is a schematic diagram showing a structure of a third embodiment of a portable blood pressure detection module of the present application;

FIG. 8 is a schematic diagram showing a structure of a fourth embodiment of a portable blood pressure detection module of the present application;

FIG. 9 is a schematic diagram showing a structure of a fifth embodiment of a portable blood pressure detection module of the present application;

FIG. 10 is a schematic diagram showing a structure of a sixth embodiment of a portable blood pressure detection module of the present application;

FIG. 11 is a schematic diagram showing a usage of the sixth embodiment of a portable blood pressure detection module of the present application;

FIG. 12 is a schematic diagram showing a usage of the seventh embodiment of a portable blood pressure detection module of the present application;

FIG. 13 is an exploded view of the embodiment shown in FIG. 12;

FIGS. 14 and 15 are a sectional view of the embodiment shown in FIG. 12.

### DETAILED DESCRIPTION OF THE INVENTION

The present application will be further described in detail below with reference to the accompanying drawings, in which like elements in different embodiments are indicated with like reference numerals. In the following embodiments, many details are described so that the present application will be better understood. However, those skilled in the art can readily recognize that some of the features may be omitted, or replaced by other elements, materials, or methods, depending on different situations. In some cases, some operations related to the present application are not shown or described in this specification, so as to avoid overwhelming the core part of the present application with excessive description. Detailed description of these relevant operations is not necessary for those skilled in the art, who can have a complete knowledge of the relevant operations in light of the description in the specification and the general technical knowledge in the art.

Additionally, the characteristics, operations or features described in the specification can be combined in any suitable manner to form various embodiments. Moreover, the steps or actions in the description of the method may also be switched or adjusted in sequence in a manner that is obvious to those skilled in the art. Therefore, the various sequences in the description and the drawings are merely for the purpose of clearly describing a particular embodiment and are not intended to be required, unless it is otherwise specified that a specific sequence must be followed.

The serial numbers per se, such as “first”, “second”, etc., designated herein for components, are only used for distinguishing the described objects and do not represent any sequence or have any technical meaning. As used herein, “connected” or “coupled” includes both direct and indirect connection (coupling), unless otherwise specified.

### Example 1

The present example provides a portable blood pressure detection module. The portable blood pressure detection module can be used alone or by attaching to other wearable devices for achieving blood pressure detection by oscillography or auscultation method.

In an implementation, the portable blood pressure detection module includes a housing, a pulse pressure transmitting member, an inflating and deflating structure, a detection unit, and a blood pressure calculation unit.

The housing has a mounting chamber, and the pulse pressure transmitting member is mounted onto the housing to form a pulse pressure transmitting structure together with the housing. It should be noted that the pulse pressure transmitting structure can not only be applied to the portable blood pressure detection module in the present example, but also to other forms of electronic sphygmomanometers, such as a desktop electronic sphygmomanometer, a watch-type electronic sphygmomanometer, and the like.

Specifically, in the pulse pressure transmitting structure, the pulse pressure transmitting member combines with the housing to form a cavity for inflation. At least a part of the pulse pressure transmitting member protrudes out of the housing, such that the pulse pressure transmitting member can, during inflation, directly or indirectly act on a body part to be measured of a subject to be detected.

The pulse pressure transmitting structure replaces a conventional airbag structure. Generally, the pulse pressure transmitting member at least partly has a certain degree of expandability or elasticity. For example, in an implementation, the pulse pressure transmitting member includes an elastic portion made of an elastic material and/or a flexible portion made of a flexible material. When in use, the cavity is inflated to expand the pulse pressure transmitting member, such that at least a part of the pulse pressure transmitting member bulges out of the housing and directly or indirectly presses against the body part to be measured, thereby achieving transmission of pulse pressure.

The inflating and deflating structure is in communication with the cavity, and is configured to inflate and deflate the cavity. The detection unit is configured to detect a pulse pressure signal in the cavity and transmit the signal to the blood pressure calculation unit, and the blood pressure calculation unit calculates a blood pressure detection result based on the detected pulse pressure signal.

The pulse pressure transmitting member may be directly pressed against the body part to be measured, or may be indirectly pressed against the body part to be measured, with another member such as a protective cover or an outer coat or the like in between.

The inflating and deflating structure, the detection unit and the blood pressure calculation unit are amounted in the mounting chamber, and the pulse pressure transmitting member can be partly or completely mounted in the mounting chamber, with only a part thereof bulging out of the housing during inflation to be pressed against the body part to be measured. Alternatively, the pulse pressure transmitting member is directly pressed against an outer wall of the housing, and the entire pulse pressure transmitting member is exposed outside the housing. The volume of the pulse pressure transmitting member is basically the same as or slightly larger than that of the housing. There is no need to wrap around a whole wrist or arm as in the case with a conventional airbag. As a result, the entire portable blood pressure detection module can be designed to be very small in size.

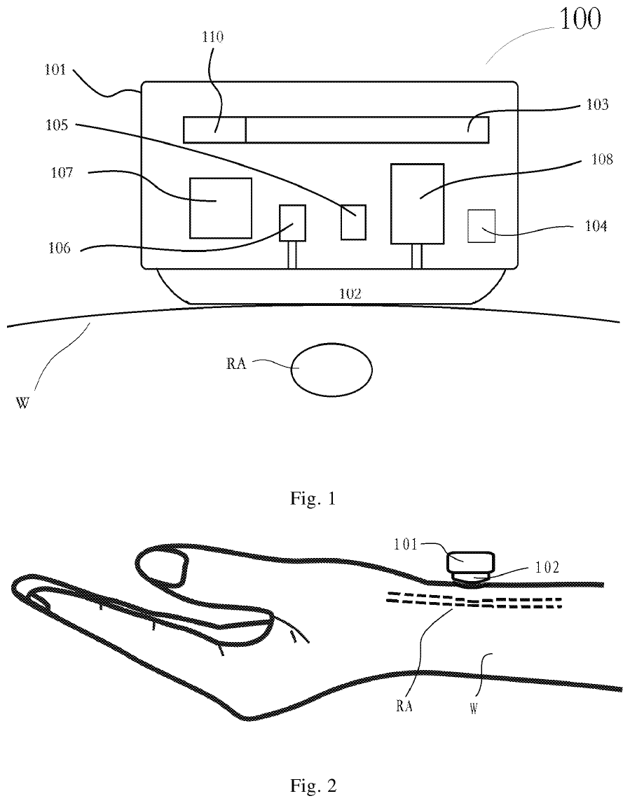

With reference to FIG. 1, in an implementation, the blood pressure calculation unit 103 may be a single-chip microcomputer or another devices having a logic operation function. The inflating and deflating structure includes a slow vent valve 104, a fast vent valve 105, and an air pump 108. The air pump 108, the slow vent valve 104, and the fast vent valve 105 are all disposed in the mounting chamber of the housing.

In this implementation, an oscillometric method is used to achieve blood pressure measurement. Specifically, the detection unit uses an air pressure sensor 106, which is disposed in the mounting chamber. In another implementation, the detection unit may also use a sound sensor to detect blood pressure by auscultation.

The pulse pressure transmitting member includes an elastic film 102, which is disposed below the housing 101 and forms a cavity together with an outer wall of the housing 101. The cavity is in communication with the air pump 108, the fast vent valve 105, and the air pressure sensor 106 to form an air path.

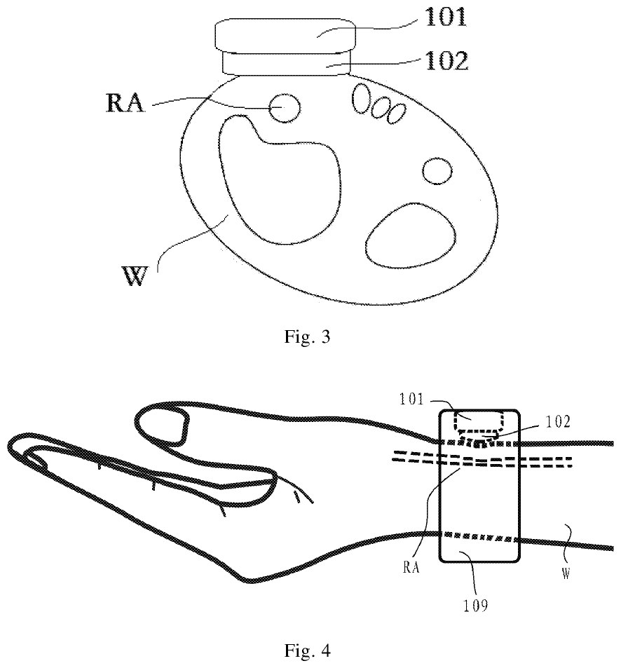

With reference to FIGS. 1-3, in an implementation, detection of wrist radial artery is taken as an example, wherein RA represents radial artery, W represents wrist, and 100 represents a portable blood pressure detection module.

When in use, the housing 101 is placed at the radial artery, and the portable blood pressure detection module 100 is pressed against the radial artery RA. At the beginning of the detection, the air pump 108 pressurizes the cavity, causing the elastic film 102 to compress the radial artery RA and obstruct blood flow. Then, the slow vent valve 104 slowly releases the compressed gas in the cavity. A change in the blood vessel pressure adds to the internal air pressure of the cavity, and the air pressure sensor 106 detects a change in the pressure in the cavity. The blood pressure calculation unit 103 calculates a blood pressure value based on a signal detected by the air pressure sensor 106. Thereafter, the fast vent valve 105 quickly vents the gas in the cavity, loosening the artery.

In addition, the portable blood pressure detection module 100 may further include a power source 107 for supplying power to electrical components. Alternatively, it may also have a battery compartment for installing a removable battery such as a dry battery. In addition, it can also have a power cable for connecting to an external power source.

The portable blood pressure detection module 100 may further include a communication unit, which is connected to the blood pressure calculation unit 103 and is configured to establish a communication connection between the portable blood pressure detection module 100 and other devices. The communication connection may be wireless or wired communication. It is preferable to select a wireless mode, which is convenient for the use of the portable blood pressure detection module 100.

With further reference to FIG. 1, in an implementation, the communication unit uses a wireless transmission unit 110, which can establish wireless communication with other terminals, such as a telecontroller, a mobile phone, a smart watch, and the like. In this way, operations of the portable blood pressure detection module 100, such as start, pause, and end, can be controlled by an external terminal, and the detection results of the portable blood pressure detection module 100 can also be displayed by an external terminal.

For example, the portable blood pressure detection module 100 starts detection after receiving a start instruction from an external terminal. After the detection is completed, the detection results are displayed on the external terminal.

On the other hand, in an implementation, the portable blood pressure detection module 100 further includes a display unit and/or a sound prompting unit, the display unit and/or the sound prompting unit being connected to the blood pressure calculation unit 103 for displaying and/or prompting the detection results.

The display unit may be a screen display device such as a display screen or a touch screen, or it may be an indicator light (indicator lights of different colors representing different detection results), or the like.

The sound prompting unit may be any device capable of sound prompting, such as voice broadcasting, or the like.

Further, the portable blood pressure detection module 100 can be independently applied to any part of the body where arterial pulse can be detected, such as a wrist, an arm, a cervical artery, the heart, and the like. When in use, the portable blood pressure detection module 100 can be fixed to these body parts by a built-in wearing structure, which is fixedly connected (directly or indirectly fixedly connected) to the housing 101 for wearing the portable blood pressure detection module 100 at an arterial blood vessel of a subject to be detected. For example, in some implementations, the wearing structure includes at least one of a strap structure, a ring structure, a suction structure, and an adhesive structure. With reference to FIG. 4, in an implementation, the wearing structure may be a wrist strap 109.

The strap structure includes a watch strap or other strap-like structures, which form a ring structure by means of buckling, binding, bonding, etc., so that the portable blood pressure detection module 100 can be worn on a wrist, a watch, or the neck, etc.

The ring structure includes an elastic ring sleeve or a ring strap structure. The suction structure includes a structure that achieves installation by means of suction, such as a sucking disk, or the like. The adhesive structure includes a structure used for adhesive fixation, such as a medical adhesive tape, or the like.

In addition, the portable blood pressure detection module 100 can be provided with no wearing structure. Rather, the portable blood pressure detection module 100 can be directly mounted by other external fixing means. For example, the portable blood pressure detection module 100 can be affixed to a body part to be detected by using a medical adhesive tape commonly used in medical treatment.

On the other hand, in addition to being used independently, the portable blood pressure detection module 100 can also be used by being attached to other wearable devices.

In other implementations, the portable blood pressure detection module 100 includes a mounting structure that is fixedly connected to the housing 101 and is used to mount the portable blood pressure detection module 100 to other devices. It is usually detachably fixed to other wearable devices. Of course, in some implementations, it may also be non-detachably fixed.

In some implementations, the mounting structure includes at least one of an adhesion structure, a snap-in structure, a screw-in structure, and a magnetic adsorption structure.

The adhesion structure includes a series of structures that can achieve mounting by adhesion, such as Velcro. The snap-in structure includes a structure that achieves fixing by a snap fit. The screw-in structure includes a structure that achieves mounting by fixing with a screw, and a structure for fastening a self screw of the portable blood pressure detection module 100 to a wearable device. The magnetic adsorption structure includes a structure that achieves fixing by utilizing a magnetic adsorption force.

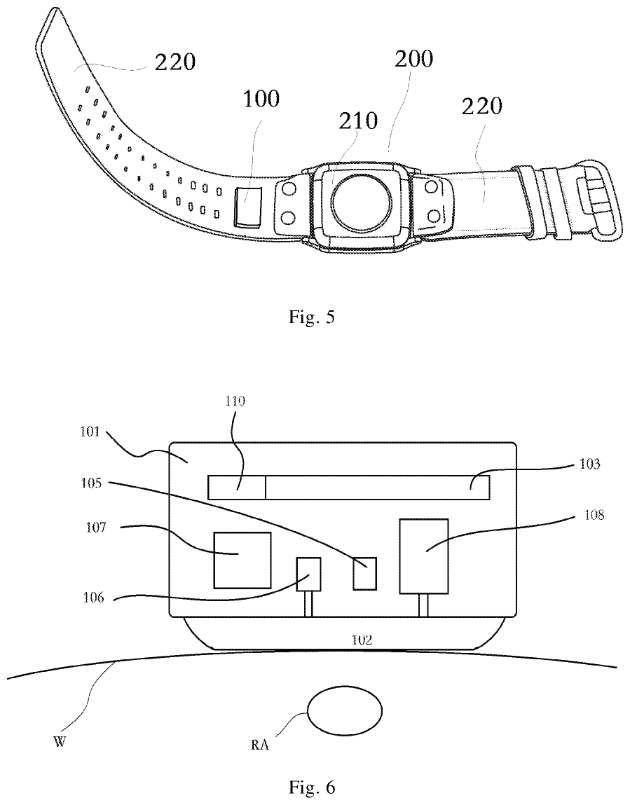

With reference to FIG. 5, in an implementation, the portable blood pressure detection module 100 is mounted on a watch 200. The watch 200 includes a watch head 210 and a watch strap 220. The portable blood pressure detection module 100 is mounted at an inner side of the watch strap 220, such that after the watch strap 220 is worn, the portable blood pressure detection module 100 can be pressed against a radial artery to measure pulse pressure.

The watch 200 may be an ordinary watch 200 for displaying time, or a smart watch 200 having other functions besides displaying time. When the watch 200 is a smart watch 200, the smart watch 200 may establish a wireless and/or wired communication connection with a communication unit of the portable blood pressure detection module 100, and then be used to control the portable blood pressure detection module 100 and/or display the test results of the portable blood pressure detection module 100.

On the other hand, in order to form a desired cavity, in some implementations, at least one of the housing 101 and the pulse pressure transmitting member has a recess, the housing 101 and the pulse pressure transmitting member closing the recess to form the above-mentioned cavity.

Of course, in other implementations, when the pulse pressure transmitting member itself is elastic, a recess needs not be formed, and the desired cavity can be formed by deformation of the material itself upon inflation.

Further, in some implementations, the pulse pressure transmitting member is mounted in the housing 101, and a part of the pulse pressure transmitting member protrudes from an inside of the housing 101 to an outside of the housing 101. In this case, only the part of the pulse pressure transmitting member that needs to be pressed against the body part to be measured protrudes from the housing 101, which allows further designing the portable blood pressure detection module 100 to be smaller and more convenient to carry and use.

Generally, the part of the pulse pressure transmitting member protruding out of the housing 101 can be made of an elastic film 102 or a flexible film. This can take advantage of the elastic characteristics of the elastic film 102 to allow the pulse pressure transmitting member to protrude out of the housing 101 when it expands and deforms, and retract into the housing 101 or to a position close to the housing 101 when it is deflated, making the portable blood pressure detection module 100 smaller in size.

With reference to FIGS. 1 and 2 again, generally, the inner diameter of a radial artery of a wrist is approximately 2.3+/−0.4 mm. Since there may be an error in positioning during wearing, a pulse pressure transmitting member can be designed to be able to completely cover the radial artery, such as an elastic film of approximately 10 mm long and 8 mm wide. The blood pressure of the radial artery can be measured by pressurizing (or depressurizing after pressurizing) the cavity to add a change in the radial artery pressure to the internal pressure of the cavity, and then measuring a change in the cavity pressure added with radial artery internal pressure.

This blood pressure detection module does not need to be wrapped around a limb, and needs only to cover part of an arterial blood vessel of the limb, which does not constitute a wearing burden. For example, as shown in FIG. 5, such device is disposed between a watch strap and a wrist radial artery to perform blood pressure detection. The size of this blood pressure detection module can even be 20 mm×15 mm×8 mm or smaller. The width of the watch strap of existing watches is generally about 20 mm, or about 30 mm if it is wider. Since the blood pressure detection module does not take up too much space on the wrist, it can be used with a watch that detects heart rate by photoelectric method.

### Example 2

The present example provides a second portable blood pressure detection module 100.

With reference to FIG. 6, the portable blood pressure detection module 100 of the present example is different from the portable blood pressure detection module 100 shown in Example 1 in that:

The inflating and deflating structure of the portable blood pressure detection module 100 of the present example includes an air pump 108 and a fast vent valve 105. The air pump 108 and the fast vent valve 105 are both built into the mounting chamber of the housing 101. The air pump 108 and the fast vent valve 105 are in communication with the cavity to form an air path.

Further with reference to FIG. 6, in an implementation, detection of wrist radial artery is taken as an example, wherein RA represents radial artery, W represents wrist, and 100 represents portable blood pressure detection module 100.

The housing 101 is placed at the radial artery RA, and the portable blood pressure detection module 100 is pressed against the radial artery RA. The air pump 108 pressurizes the cavity, causing the elastic film 102 to compress the radial artery RA, such that a change in the blood vessel pressure adds to the internal air pressure of the cavity. At the same time, the air pressure sensor 106 detects a change in cavity pressure, and the blood pressure calculation unit 103 calculates a blood pressure value based on a signal detected by the air pressure sensor 106. Thereafter, the fast vent valve 105 quickly vents the gas in the cavity.

### Example 3

The present example provides a third portable blood pressure detection module 100.

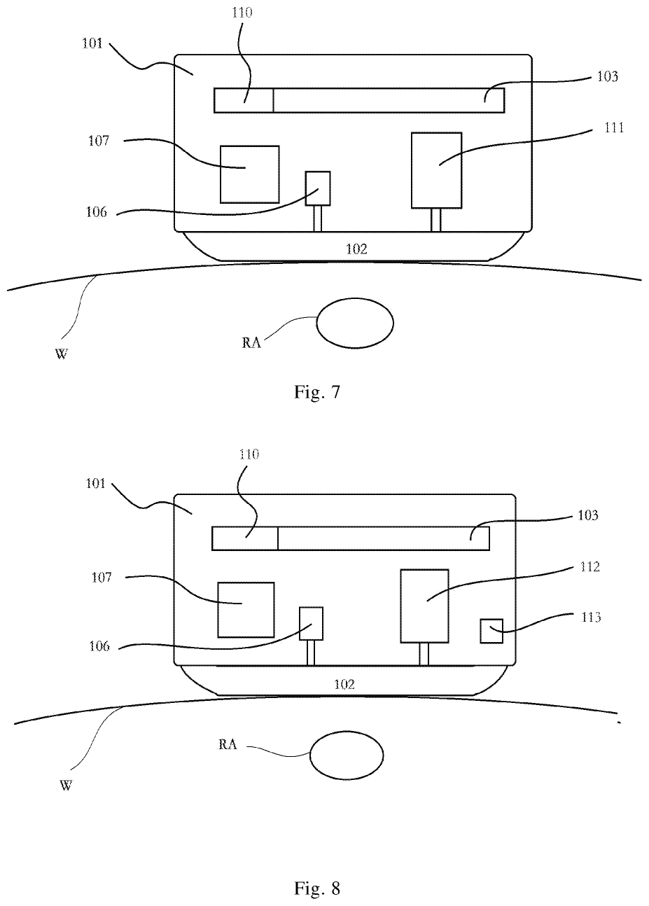

With reference to FIG. 7, the portable blood pressure detection module 100 of the present example is different from the portable blood pressure detection module 100 shown in Example 2 in that:

The inflating and deflating structure of the portable blood pressure detection module 100 of the present example includes an integrated air pump 111 having an inflating and deflating function, and the integrated air pump 111 is in communication with the cavity to form an air path. The integrated air pump 111 is built into a mounting chamber of the housing 101.

With reference to FIG. 7, in an implementation, detection of wrist radial artery is taken as an example, wherein RA represents radial artery, W represents wrist, and 100 represents portable blood pressure detection module 100.

The housing 101 is placed at the radial artery RA, and the portable blood pressure detection module 100 is pressed against the radial artery RA. The integrated air pump 111 pressurizes the cavity, causing the elastic film 102 to compress the radial artery RA, such that a change in the blood vessel pressure adds to the internal air pressure of the cavity. At the same time, the air pressure sensor 106 detects a change in cavity pressure, and the blood pressure calculation unit 103 calculates a blood pressure value based on a signal detected by the air pressure sensor 106. Thereafter, the gas in the cavity is quickly vented through the integrated air pump 111.

### Example 4

The present example provides a fourth portable blood pressure detection module 100.

With reference to FIG. 8, the portable blood pressure detection module 100 of the present example is different from the portable blood pressure detection module 100 shown in Example 3 in that:

The detection unit of the portable blood pressure detection module 100 of the present example includes a sound sensor 113 and an air pressure sensor 106. The sound sensor 113 is used to detect Coriolis sound, and the air pressure sensor 106 detects a change in cavity pressure.

On the other hand, the inflating and deflating structure of the portable blood pressure detection module 100 of the present example includes a pressure-increasing and pressure-reducing air pump 112, which is built into a mounting chamber of the housing 101, and is in communication with the cavity to form an air path.

With reference to FIG. 8, in an implementation, detection of wrist radial artery is taken as an example, wherein RA represents radial artery, W represents wrist, and 100 represents portable blood pressure detection module 100.

The housing 101 is placed at the radial artery RA, and the portable blood pressure detection module 100 is pressed against the radial artery. The pressure-increasing and pressure-reducing air pump 112 pressurizes the cavity, causing the elastic film 102 to compress the radial artery, such that a change in the blood vessel pressure adds to the internal air pressure of the cavity. At the same time, the sound sensor 113 detects Coriolis sound, and the air pressure sensor 106 detects a change in cavity pressure. The blood pressure calculation unit 103 calculates a blood pressure value based on Coriolis sound signal and a signal detected by the air pressure sensor 106. Thereafter, the pressure-increasing and pressure-reducing air pump 112 vents the gas in the cavity.

### Example 5

The present example provides a fifth portable blood pressure detection module 100.

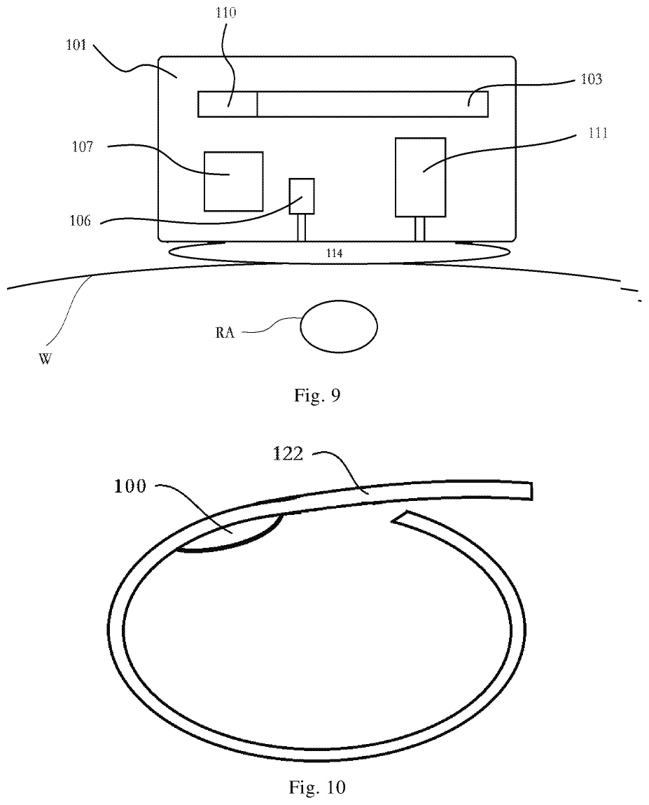

With reference to FIG. 9, the portable blood pressure detection module 100 of the present example is different from the portable blood pressure detection module 100 shown in Example 1 in that:

The pulse pressure transmitting structure of the portable blood pressure detection module 100 of the present example forms a capsule structure 114, and the pulse pressure transmitting member is closed into a cavity by the housing 101 or a component fixed on the housing 101.

### Example 6

The present example provides a six portable blood pressure detection module 100.

With reference to FIGS. 10 and 11, the portable blood pressure detection module 100 of the present example is different from the portable blood pressure detection module 100 shown in Example 1 in that:

The portable blood pressure detection module 100 of the present example has a wrist strap 122 as a wearing structure. The housing 101 of the portable blood pressure detection module 100 is mounted at an inner side of the wrist strap 122, such that when the wrist strap 122 is worn, the portable blood pressure detection module 100 can be pressed against an artery at a wrist or an arm.

### Example 7

The present example provides a seventh portable blood pressure detection module.

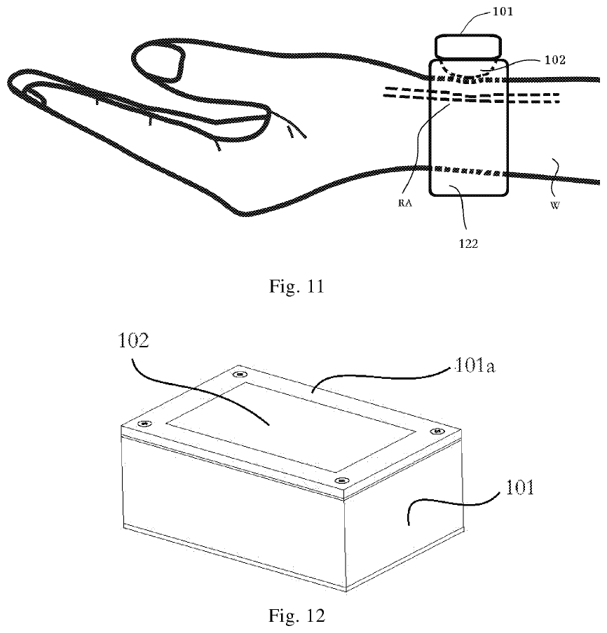

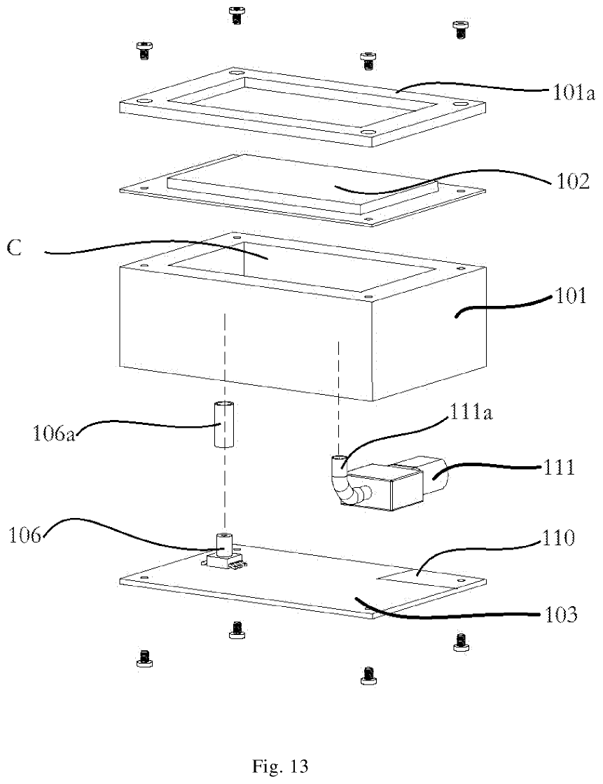

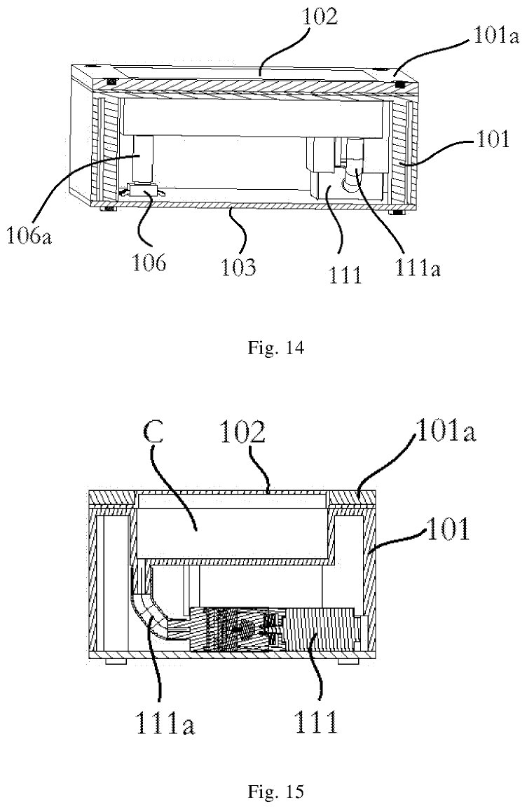

With reference to FIGS. 12 and 15, the portable blood pressure detection module of the present example is different from the portable blood pressure detection module 100 shown in Example 1 in that:

An outer wall of the housing 101 of the portable blood pressure detection module of the present example is recessed to form a first recess, and a side of the pulse pressure transmitting member 102 facing the first recess is recessed to form a second recess. The pulse pressure transmitting member 102 is pressed and sealed onto the first recess by a pressing member 101a, such that the first recess and the second recess constitute a cavity C for inflation.

The pressing member 101a is a hollow pressing plate, and a part of the pulse pressure transmitting member 102 (for example, an elastic film) is accommodated in the hollow portion of the pressing plate. The pressure plate presses the pulse pressure transmitting member 102 onto the housing 101 to form the closed cavity C.

Further with reference to FIGS. 12 and 15, a side of the housing 101 opposite to the first recess has a mounting chamber, in which the air pressure sensor 106 and the integrated air pump 111, among other components, can be mounted. The air pressure sensor 106 is in communication with the cavity C through a connecting pipe 106a, and the integrated air pump 111 is in communication with the cavity C through a connecting pipe 111a. When the integrated air pump 111 is powered on, a normally open valve is closed to conduct pressurization, and after the power is turned off, pressurization is stopped and the normally open valve is opened.

### Example 8

The present example provides a smart bracelet that includes the pulse pressure transmitting structure shown in any one of the above examples for conducting pulse pressure of a subject.

In addition, the smart bracelet also includes an inflating and deflating structure, a detection unit, a blood pressure calculation unit, a display unit and/or a sound prompting unit.

The inflating and deflating structure is configured to inflate and deflate the cavity. The detection unit is configured to detect a pulse pressure signal in the cavity. The blood pressure calculation unit is configured to calculate a blood pressure detection result based on the detected pulse pressure signal. The display unit and/or the sound prompting unit are/is connected to the blood pressure calculation unit, and are/is configured to display and/or prompt the detection result.

The inflating and deflating structure, the detection unit, the blood pressure calculation unit, the display unit and/or the sound prompting unit may adopt the solutions disclosed in any of the above examples, or may use other solutions not described in the above examples.

The pulse pressure transmitting structure is usually disposed on a ring of a smart bracelet to enable the pulse pressure transmitting structure to be pressed against an arterial blood vessel.

In addition, the smart bracelet can also have other added functions, such as other vital sign parameters, step detection, sleep quality, heart rate, among other functions.

### Example 9

The present example provides a smart bracelet that includes the blood pressure detection module shown in any one of the above examples for detecting pulse pressure of a subject.

The blood pressure detection module may be non-detachably mounted or detachably mounted. Usually, the blood pressure detection module can be disposed on a ring of the smart bracelet to enable the pulse pressure transmission structure to be pressed against an arterial blood vessel.

In addition, the smart bracelet can also have other added functions, such as other vital sign parameters, step detection, sleep quality, heart rate, among other functions.

### Example 10

The present example provides a smart watch that includes the pulse pressure transmission structure shown in any one of the above examples for transmitting pulse pressure of a subject.

In addition, the smart watch also includes an inflating and deflating structure, a detection unit, a blood pressure calculation unit, a display unit and/or a sound prompting unit.

The inflating and deflating structure is configured to inflate and deflate the cavity. The detection unit is configured to detect a pulse pressure signal in the cavity. The blood pressure calculation unit is configured to calculate a blood pressure detection result based on the detected pulse pressure signal. The display unit and/or the sound prompting unit are/is connected to the blood pressure calculation unit, and are/is configured to display and/or prompt the detection result.

The inflating and deflating structure, the detection unit, the blood pressure calculation unit, the display unit and/or the sound prompting unit may adopt the solutions disclosed in any of the above examples, or may use other solutions not described in the above examples.

The pulse pressure transmitting structure is usually disposed on a part of an inner side of a watch strap of the smart watch that is adjacent to an arterial blood vessel to enable the pulse pressure transmitting structure to be pressed against the arterial blood vessel.

In addition, the smart bracelet can also have other added functions, such as other vital sign parameters, step detection, sleep quality, heart rate, among other functions.

### Example 11

The present example provides a smart watch that includes the blood pressure detection module shown in any one of the above examples for detecting pulse pressure of a subject.

The blood pressure detection module may be non-detachably mounted or detachably mounted. Usually, the blood pressure detection module can be disposed on a part of an inner side of a watch strap of the smart watch that is adjacent to an arterial blood vessel to enable the pulse pressure transmission structure to be pressed against the arterial blood vessel.

In addition, the smart bracelet can also have other added functions, such as other vital sign parameters, step detection, sleep quality, heart rate, among other functions.

Besides being able to be applied to the smart watches and smart bracelets described above, the pulse pressure transmission structure and blood pressure detection module shown above can also be applied to other smart wearable devices by reference to the above examples for the mode of application. Smart wearable devices refer to apparatuses and devices with data processing capabilities that can be worn on a human body.

The present invention has been described above with reference to specific examples, which are merely intended to aid the understanding of the present invention and are not intended to limit the present invention thereto. Several simple derivations, variations or substitutions can be made by a person skilled in the art to which the present invention pertains in light of the concept of the present invention.

1. A pulse pressure transmitting structure for an electronic sphygmomanometer, comprising a housing and a pulse pressure transmitting member, the pulse pressure transmitting member being mounted on the housing and joined with the housing to form a cavity for inflation, and at least a part of the pulse pressure transmitting member protruding out of the housing, such that the pulse pressure transmitting member can, during inflation, directly or indirectly act on a body part to be measured of a subject to be detected.

2. The pulse pressure transmitting structure according to claim 1, wherein the pulse pressure transmitting member comprises an elastic portion made of an elastic material and/or a flexible portion made of a flexible material.

3. The pulse pressure transmitting structure according to claim 1, wherein at least one of the housing and the pulse pressure transmitting member has a recess, the housing and the pulse pressure transmitting member closing the recess to form the cavity.

4. The pulse pressure transmitting structure according to claim 3, wherein an outer wall of the housing is recessed to form a first recess, and a side of the pulse pressure transmitting member facing the first recess is recessed to form a second recess, the pulse pressure transmitting member being pressed and sealed onto the first recess by a pressing member, such that the first recess and the second recess constitute the cavity for inflation.

5. The pulse pressure transmitting structure according to claim 1, wherein the pulse pressure transmitting member is mounted in the housing, and a part of the pulse pressure transmitting member protrudes from an inside of the housing to an outside of the housing.

6. The pulse pressure transmitting structure according to claim 1, wherein the part of the pulse pressure transmitting member protruding out of the housing is made of an elastic film or a flexible film.

7. A portable blood pressure detection module, comprising:
a housing having a mounting chamber;
a pulse pressure transmitting member mounted on the housing and joined with the housing to form a cavity for inflation, at least a part of the pulse pressure transmitting member protruding out of the housing, such that the pulse pressure transmitting member can, during inflation, directly or indirectly act on a body part to be measured of a subject to be detected;
an inflating and deflating structure in communication with the cavity and configured to inflate and deflate the cavity;
a detection unit configured to detect a pulse pressure signal in the cavity;
and a blood pressure calculation unit configured to calculate a blood pressure detection result based on the detected pulse pressure signal;
the inflating and deflating structure, the detection unit and the blood pressure calculation unit being mounted in the mounting chamber.

8. The portable blood pressure detection module according to claim 7, further comprising a communication unit, the communication unit being connected to the blood pressure calculation unit and configured to establish a communication connection between the portable blood pressure detection module and other devices.

9. The portable blood pressure detection module according to claim 7, further comprising a display unit and/or a sound prompting unit, the display unit and/or the sound prompting unit being connected to the blood pressure calculation unit and configured to display and/or prompt a detection result.

10. The portable blood pressure detection module according to claim 7, wherein the inflating and deflating structure comprises an air pump configured for pressurizing and a fast vent valve configured for venting, the air pump and the fast vent valve being in communication with the cavity.

11. The portable blood pressure detection module according to claim 7, wherein the inflating and deflating structure comprises an integrated air pump having an inflating and deflating function, the integrated air pump being in communication with the cavity.

12. The portable blood pressure detection module according to claim 7, further comprising a mounting structure, the mounting structure being fixedly connected to the housing and configured to mount the portable blood pressure detection module to other devices.

13. The portable blood pressure detection module according to claim 7, further comprising a wearing structure, the wearing structure being fixedly connected to the housing and configured to wear the portable blood pressure detection module at an arterial blood vessel of a subject to be detected.

14. The portable blood pressure detection module according to claim 7, wherein the pulse pressure transmitting member comprises an elastic portion made of an elastic material and/or a flexible portion made of a flexible material.

15. The blood pressure detection module according to claim 14, wherein at least one of the housing and the pulse pressure transmitting member has a recess, the housing and the pulse pressure transmitting member closing the recess to form the cavity.

16. The portable blood pressure detection module according to claim 15, wherein an outer wall of the housing is recessed to form a first recess, and a side of the pulse pressure transmitting member facing the first recess is recessed to form a second recess, the pulse pressure transmitting member being pressed and sealed onto the first recess by a pressing member, such that the first recess and the second recess constitute the cavity for inflation.

17. The portable blood pressure detection module according to claim 14, wherein the pulse pressure transmitting member is mounted in the housing, and a part of the pulse pressure transmitting member protrudes from an inside of the housing to an outside of the housing.

18. The blood pressure detection module according to claim 17, wherein the part of the pulse pressure transmitting member protruding out of the housing is made of an elastic film or a flexible film

19. A smart wearable device, comprising a blood pressure detection module according to claim 7, configured to detect a pulse pressure of a subject to be detected.

20. The smart wearable device according to claim 19, further comprising:
a second communication unit configured to establish a wireless and/or wired communication connection with the blood pressure detection module;
and a display unit and/or a sound prompting unit configured to display and/or prompt a detection result.
